(19) United States
(12) Patent Application Publication
(10) Pub. No.: US 2006/0246536 A1
(43) Pub. Date: Nov. 2, 2006

(54) Xenograft tissue control for histology

(76) Inventors: Margaret Hardy, Tucson, AZ (US); Thomas M. Grogan, Tucson, AZ (US); Ray B. Nagle, Tucson, AZ (US)

Correspondence Address: VENTANA MEDICAL SYSTEMS, INC.;ATTENTION: LEGAL DEPARTMENT, 1910 INNOVATION PARK DRIVE, TUCSON, AZ, 85755, US

(21) Appl. No.: 11/416,362

(22) Filed: May 1, 2006

Related U.S. Application Data
(60) Provisional application No. 60/676,056, filed on Apr. 29, 2005.

(51) Int. Cl. G01N 1/30; C12N 5/06; C12N 5/08

(52) U.S. Cl. 435/40.5; 435/354; 435/367

(57) ABSTRACT

An embodiment of the invention is a method of using a xenograft as a control tissue for histology, comprising staining both a patient and a xenograft-derived control sample under substantially similar staining conditions, and assessing the staining outcomes of the two to determine whether the stain was effective for the patient sample. A xenograft has never been used before in histology as a control, as far as the inventors know. The result of using a xenograft as a control is surprisingly advantageous. First, the cell lines grow and differentiate similarly to a human, taking on the general morphology of a real tissue sample. Second, because the same transformed cell line can be grown limitless times in SCID mice, the xenograft control is highly reproducible, leading to a consistent artificial control that is highly manufacturable and subject to genetic manipulation so that antigens or genetic elements may be embedded in the tissue. Another embodiment of the invention is directed generally to a method of making a tissue control substrate, comprising growing a xenograft from a mammalian transformed cell line in a host animal, removing the xenograft from the host animal, processing the xenograft thereby embedding the xenograft tissue in an embedding medium, and finally affixing the embedded xenograft sample onto a substrate. The substrate is generally a microscope slide. The xenograft control slide can then be stained side-by-side with a specimen sample in an automated slide stainer, and act as a control against which the staining quality can be compared. The xenograft control can also be used as a manual staining control. Determining whether the staining was effective for the patient specimen comprises judging the staining intensity of the xenograft control sample to determine if the expected degree and type of staining were realized in the control. If the expected type (nuclear, membranous, or cytoplasmic) and degree (0-4 scale) of staining are realized during the run, then the xenograft control indicates the staining process and reagents are working properly, and so the result in the patient specimen can be trusted. A further embodiment of the invention is a xenograft-derived control slide for histochemical use, comprising at least one xenograft control sample prepared for histological use, and a sample slide upon which the at least one xenograft control sample is affixed.

### CROSS-REFERENCE TO RELATED CASES

This application claims priority to U.S. Provisional patent application Ser. No. 60/676,056 filed Apr. 29, 2005, the entire contents of which are incorporated by reference herein.

### BACKGROUND

1. Field of the Invention

The invention is directed generally to the area of Histology, particularly the area of tissue controls for histochemical processing of patient samples.

2. Description of Related Art

The use of controls in Histology is essential. Controls are used in all three sub-disciplines including Immunohistochemistry (“IHC”), in situ hybridization (“ISH”) and special stains (“SS”). Traditionally, two types of controls are used, positive and negative. Tissue controls are usually created from either the patient tissue block that is to be run, or from a known, well-characterized archival tissue block.

In IHC, positive control testing is performed on sections of tissue known to contain the target antigen, processed using the same fixation, epitope retrieval and immunostaining protocols as the patient tissue. A separate tissue section may be used as a positive control, but test sections often contain normal elements that express the antigen of interest (internal controls). Internal positive controls are acceptable for these antigens, but unless the internal control tissue is very-well characterized, there is always some concern that the internal control may not be reliable.

A positive control section included on the same slide as the patient tissue is optimal practice because it helps to identify any failure to apply primary antibody or other critical reagent to the patient test slide; however, one separate positive control per staining run for each antibody in the run (batch control) may be sufficient provided that the control slide is closely scrutinized by a qualified reviewer.

Ideally, positive control tissues possess low levels of antigen expression, as is often seen in neoplasms, and/or infectious diseases. Exclusive use of normal tissues that have high levels of antigen expression may result in antibody titers (concentrations) of insufficient sensitivity, leading to false-negative results.

A negative reagent control is used to assess nonspecific staining in patient tissue. A separate section of patient tissue is processed using the same reagent and epitope retrieval protocol as the patient test slide, except that the primary antibody is omitted, and replaced by any of the following: an unrelated antibody of the same isotype as the primary antibody (for monoclonal primary antibodies); an unrelated antibody from the same animal species as the primary antibody (for polyclonal primary antibodies); or the negative control reagent included in the staining kit.

A separate negative reagent control should be run for each block of patient tissue being immunostained.

The negative reagent control would ideally control for each reagent protocol and antibody retrieval condition; however, large antibody panels often employ multiple antigen retrieval procedures. In such cases, a reasonable minimum control would be to perform the negative reagent control using the most aggressive retrieval procedure in the particular antibody panel.

It is also important to assess the specificity of each antibody by a negative tissue control, which must show no staining of tissues known to lack the antigen. The negative tissue control is processed using the same fixation, epitope retrieval and immunostaining protocols as the patient tissue. Unexpected positive staining of such tissues indicates that the test has lost specificity, perhaps because of improper antibody concentration or excessive antigen retrieval. Intrinsic properties of the test tissue may also be the cause of “non-specific” staining. For example, tissues with high endogenous biotin activity such as liver or renal tubules may simulate positive staining when using a detection method based on biotin labeling.

One can use a separate negative tissue control slide for each antibody, or the patient test slide itself may be used for this purpose if it contains appropriate tissue components known to lack the target antigens. Multitissue control blocks may serve as both positive and negative tissue controls. Leong A S,. Cooper K, Leong F J. Manual of Diagnostic Antibodies for Immunohistology. London: Greenwich Medical Media; 1999; Dabbs D J., Diagnostic Immunohistochemistry, New York, Churchill Livingstone (2002); Burry R W, Specificity controls for immunocytochemical methods, J. Histochem. Cytochem. 48:163-166 (2000); Weirauch M., Multitissue control block for immunohistochemistry, Lab. Med. 30:448-449 (1999); O'Leary T J, Standardization in immunohistochemistry, Appl. Immunohistochem. Molecul. Morphol. 9:3-8 (2001).

Negative tissue controls are tissues known not to express the antigen of interest, and so should show no staining if the staining assay is functioning correctly. However, in both positive and negative controls, since the tissue used is non-standardized, it is never fully characterized and so there is always some doubt as to the control's usefulness.

Severe Combined Immune-deficient (“SCID”) mice are a strain of mouse that is mutated to be deficient in both T- and B-cell production. This deficiency allows them to be a ‘host’ for a wide variety of biological materials that their immune system would have normally rejected. Since its discovery by Bosma et al. in 1983 (Nature 301(5900): 527-30), the SCID mouse has been used extensively to create in vivo therapeutic tissue models both to understand the biology of diseases and also to evaluate treatments. One of the primary models is solid tumor growth, especially tumors of human origin. These tumors are, like the cell lines themselves, human in origin (genetically) and develop similarly to actual tumors in human patients; however, there are several advantages to tissue produced in this manner. First, the tumors have more homogenous characteristics because they are grown under controlled conditions from transformed cell lines that are derived from a single source and are not under the same selective pressures that tumors in a normal host are subjected to. Secondly, the cell lines are typically well described and have many characteristics defined. Thirdly, the ‘same’ tumor can be grown multiple times providing a highly consistent sample. Finally, there are no issues with regard to patient confidentiality or consent. Cell lines may also be immortalized by an infectious agent such as HPV (Human papillomavirus), the cause of nearly all cervical cancers.

What is needed is a better tissue control that is more manufacturable, highly characterized, and subject to genetic manipulation so that it can be optimized for any control situation.

### SUMMARY OF THE INVENTION

An embodiment of the invention is a method of using a xenograft as a control tissue for histology, comprising staining both a patient and a xenograft-derived control sample under substantially similar staining conditions, and assessing the staining outcomes of the two to determine whether the stain was effective for the patient sample. A xenograft has never been used before in histology as a control, as far as the inventors know. The result of using a xenograft as a control is surprisingly advantageous. First, the cell lines grow and differentiate similarly to a human, taking on the general morphology of a real tissue sample. Second, because the same transformed cell line can be grown limitless times in SCID or other immunodeficient mice, the xenograft control is highly reproducible, leading to a consistent artificial control that is highly manufacturable.

Another embodiment of the invention is directed generally to a method of making a tissue control substrate, comprising growing a xenograft from a mammalian transformed cell line in a host animal, removing the xenograft from the host animal, processing the xenograft thereby embedding the xenograft tissue in an embedding medium, and finally affixing the embedded xenograft sample onto a substrate. The substrate is generally a microscope slide. The xenograft control slide can then be stained side-by-side with a specimen sample in an automated slide stainer, and act as a control against which the staining quality can be compared. The xenograft control can also be used as a manual staining control. Determining whether the staining was effective for the patient specimen comprises judging the staining intensity of the xenograft control sample to determine if the expected degree and type of staining were realized in the control. If the expected type (nuclear, membranous, or cytoplasmic) and degree (0-4 scale) of staining are realized during the run, then the xenograft control indicates the staining process and reagents are working properly, and so the result in the patient specimen can be trusted.

A further embodiment of the invention is a xenograft-derived control slide for histochemical use, comprising at least one xenograft control sample prepared for histological use, and a sample slide upon which the at least one xenograft control sample is affixed.

### BRIEF DESCRIPTION OF THE DRAWINGS

FIG. 1 is a pictorial depiction of the steps of making a xenograft tissue, starting with inoculation of the SCID mouse with human-derived transformed cells.

FIG. 2 is a pictorial depiction of the continued process of FIG. 1, starting with the coring of the xenograft block and ending with the creation of the xenograft multiblock.

FIGS. 3a and 3b show several microscope slides; in 3a are shown four slides having xenograft control tissues arrayed on them, and 3b shows two of those slides additionally having patient specimens arranged on them.

FIGS. 4a and 4b are microphotographs of an HPV xenograft control slide tested with an ISH assay using an HPV DNA probe. CaSki (a) and HeLa (b) xenograft tissues exhibit positive staining (blue punctuate pattern). Negative staining (no blue punctuate pattern) is seen in the T24 (c) xenograft.

FIG. 5 is a photomicrograph of Her-2 xenograft control slide tested with an IHC assay using anti-Her-2 rabbit monoclonal antibody 4B5. BT-474 (a) breast carcinoma xenograft tissue exhibits a positive stain (brown cytoplasmic membrane). Negative staining (no brown pattern) is seen in the MCF7 (b) xenograft tissue.

FIG. 6 is a photograph of the HPV xenograft control slide containing a patient sample and tested with an ISH assay using an HPV DNA probe. A negatively stained slide is shown on the left and exhibits no blue punctuate pattern. The slide on the right is positively stained with the INFORM® HPV Family 16 probe and exhibits a blue punctuate pattern in all samples but the negative core (C33a).

FIGS. 6A-6H are photomicrographs (20×) of the two slides from FIG. 6. FIGS. 6A-6D are the Caski, HeLa and C33a xenograft control cores, and patient specimen, respectively, from the negatively-stained left slide of FIG. 6. FIGS. 6E-6H are the Caski, HeLa, C33a and patient specimen photomicrographs of the positively-stained right slide, respectively.

FIG. 7 is a photograph of the Her-2 xenograft control slide tested with an IHC assay using anti-Her-2 rabbit monoclonal antibody 4B5. BT-474 (lower slide) breast carcinoma xenograft tissue exhibits a positive stain (brown cytoplasmic membrane).

FIGS. 7A-7F are photomicrographs (20×) of the two slides from FIG. 7. FIGS. 7A-7C are the BT474, ZR75-1 and MCF7, respectively xenograft control cores, from the negatively-stained top slide of FIG. 7. FIGS. 7D-7F are the BT474, ZR75-1 and MCF7, respectively xenograft control cores, from the positively-stained bottom slide of FIG. 7.

### DESCRIPTION OF THE PREFERRED EMBODIMENTS

An embodiment of the invention is a method of using a xenograft as a control tissue for histology, comprising staining both a patient and a xenograft-derived control sample under substantially similar staining conditions, and assessing the staining outcomes of the two to determine whether the stain was effective for the patient sample. A xenograft has never been used before in histology as a control, as far as the inventors know. The result of using a xenograft as a control is surprisingly advantageous. First, the cell lines grow and differentiate similarly to a human, taking on the general morphology of a real tissue sample. Second, because the same transformed cell line can be grown limitless times in SCID mice or other immunodeficient mouse, the xenograft control is highly reproducible, leading to a consistent artificial control that is highly manufacturable and subject to genetic manipulation so that antigens or genetic elements may be embedded in the tissue.

Another embodiment of the invention is directed generally to a method of making a tissue control substrate, comprising growing a xenograft from a mammalian transformed cell line immortalized by either a neoplastic process or an infectious agent, in a host animal, removing the xenograft from the host animal, processing the xenograft thereby embedding the xenograft tissue in an embedding medium, and finally affixing the embedded xenograft sample onto a substrate. The substrate is generally a microscope slide. The xenograft control slide can then be stained side-by-side with a specimen sample in an automated slide stainer, and act as a control against which the staining quality can be compared. The xenograft control can also be used as a manual staining control. Determining whether the staining was effective for the patient specimen comprises judging the staining intensity of the xenograft control sample to determine if the expected degree and type of staining were realized in the control. If the expected type (nuclear, membranous, or cytoplasmic) and degree (0-4 scale) of staining are realized during the run, then the xenograft control indicates the staining process and reagents are working properly, and so the result in the patient specimen can be trusted.

A further embodiment of the invention is a xenograft-derived control slide for histochemical use, comprising at least one xenograft control sample prepared for histological use, and a sample slide upon which the at least one xenograft control sample is affixed.

The following definitions are used throughout the specification and are to be given the meanings described below.

### DEFINITIONS

DNA—Deoxyribonucleic Acid. The molecular matter that encodes the genetic make-up of living organisms.
H&E—A chemical-based dye staining of tissue using Hematoxylin and Eosin which allows someone practiced in the art to differentiate morphology through microscopic examination.
Host—any biological entity capable of supporting and growing xenografts, for instance a SCID mose, nude mouse or other immunodeficient host animal.
HPV—Human papillomavirus.
IHC—immunohistochemistry. Antibody-based assay system that is used to detect the presence or absence of specific analytes (typically proteinaceous in nature) in morphologic structures for the purpose of diagnosing and/or treating pathologic conditions.
ISH—in situ hybridization. A DNA or RNA probe based assay system that is used to detect the presence or absence of specific analytes (typically genetic, transcriptive or translational in nature) in morphologic structures for the purpose of diagnosing and/or treating pathologic conditions.
Preservation/Fixation—A chemical method of treating a tissue sample to prevent decomposition once the tissue is no longer living. Examples of preservation are formalin fixation in neutral-buffered formalin or zinc-formalin or alcohol fixation in methanol or ethanol based mixtures.
Primary cell line—Cells that have been removed from a natural source which are only capable of limited growth in vitro and have not been modified to grow indefinitely.
Processing—used to describe any and all activities involved in the harvesting, preserving, embedding, coring, sectioning, mounting and preparing for testing of xenograft specimens which are currently routine practice in pathology laboratories and those which may be used in the future.
RNA—Ribonucleic Acid. The molecules that are used by biological organisms to transcribe and transfer information encoded in the DNA to the cell for processing.
Sample platform—any substrate or medium used to immobilize the tissue sample to allow testing of the tissue and its subsequent analysis. Microscope slides are typical.
SS—Special Stain. A chemical dye or combination of dyes used in staining of tissue sections to preferentially stain morphologic structures at a cellular and sub-cellular level which allows identification of normal and pathologic characteristics to those of ordinary skill in the art. Examples include Alcian Yellow, Alcian Blue, Giemsa, Congo Red, Mucicarmine, Jones Light Green, etc.
Testing—any and all methods used to determine pathologic characteristics of tissue samples through microscopic examination by those of ordinary skill in the art. Examples used today are H&E, IHC, ISH and SS. Testing can be done manually or by using automation. Typically, testing produces some type of staining or color change in the test sample. Examples are chemical dyes such as are used in H&E stains, or colorimetric assays which use enzyme-based reactions to determine the presence or absence of analytes such as DNA, RNA or proteins.
Transformed cell line—A homogeneous and/or clonal population of cells that have been modified in vitro to grow indefinitely. Modification may be due to infectious viral agent, or by a neoplastic process. They can be from mammalian sources, including for example human, rat, hamster, rabbit, mouse, etc.
Xenografts—any tissue or tissue types from another animal or species implanted into a host animal. The tissue can be from the same or different species and can be composed of but not limited to transformed and/or primary cell lines with the final form of the xenograft(s) being comprised of but not limited to a solid mass or individual cells.
   Methods

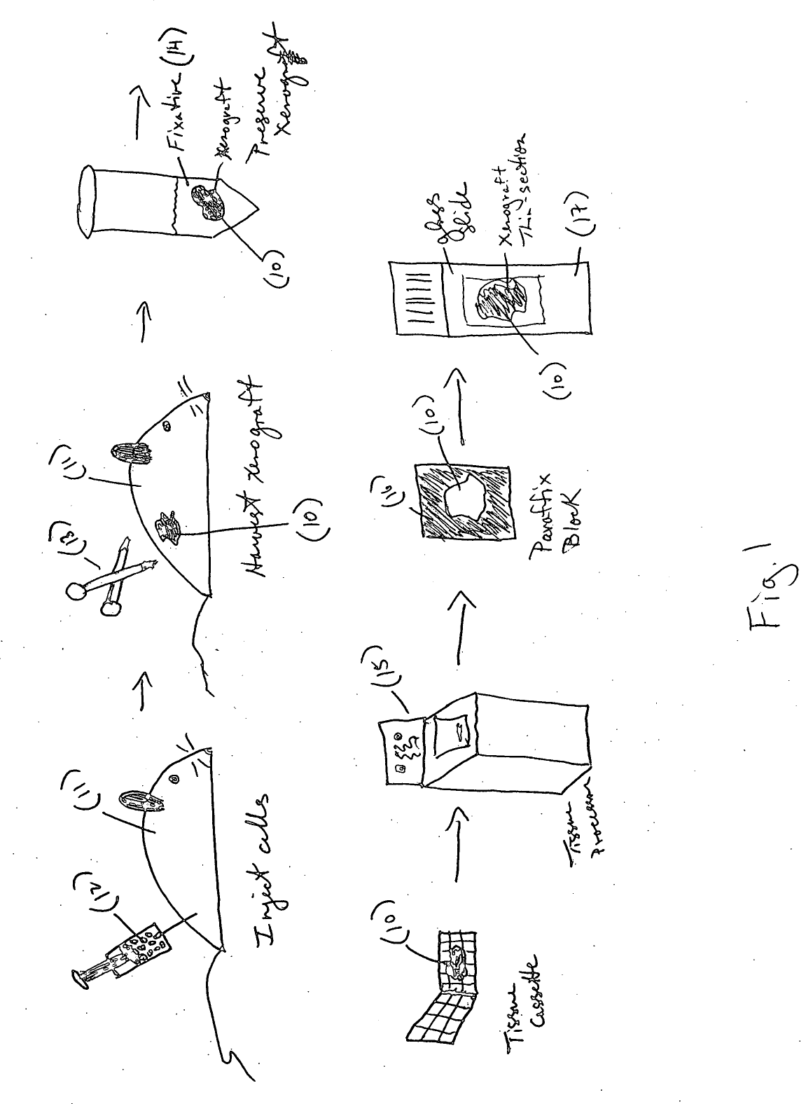

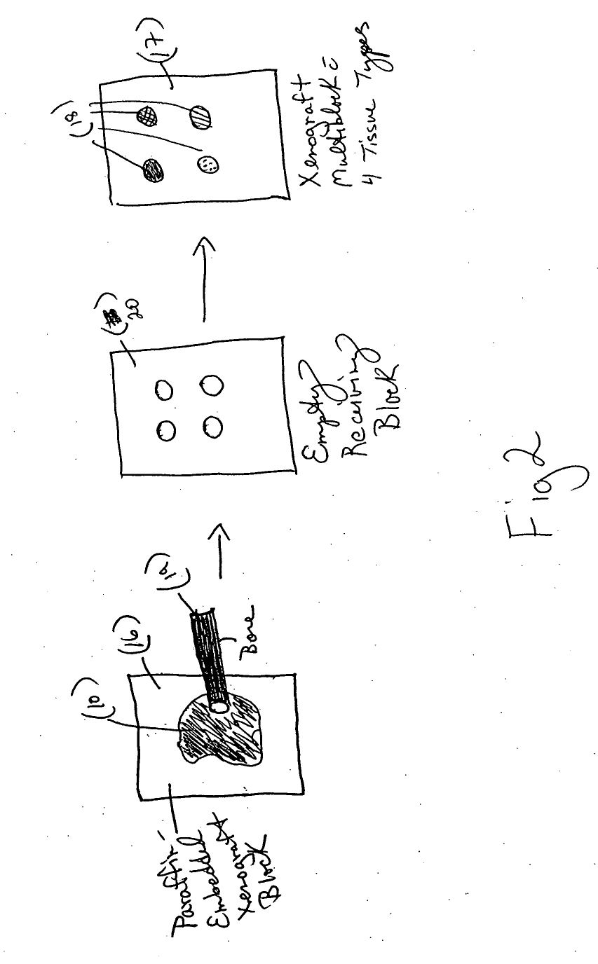

A method for creating a control sample using xenograft technology, preparing it for use with a diagnostic test and for analysis through microscopic examination is described below and is illustrated in FIGS. 1-3.

A general description of the process of making a xenograft control slide is presented in FIG. 1 to give an overview of the invention. With particular attention to the upper left portion of FIG. 1, xenograft tissues are created by injecting cells or tissues (aggregates of cells) 12 capable of growing in vivo into a host 11. The cells can be derived from a transformed cell line or may be a primary cell line. The cell line can have the same genetic background as the host animal or it may differ. This difference can be as small as a single genetic mutation or large enough that the host and xenograft are from separate species. The cell line can be comprised of more than one cell type, as long as all cell types are capable of growing under similar conditions into xenograft tissue.

A xenograft tissue 10 can be but is not limited to a solid mass of cells (tumor) or may be individual cells that are collected and aggregated together for purposes of creating the sample. An example of a solid tumor is a mass or body formed when an adherent transformed cell line such as BT-474, derived from an undifferentiated human breast carcinoma, is grown in a SCID mouse. The injected cells grow into a solid mass of tissue which is then surgically removed. An example of aggregated xenograft cells is when primary cells of lymphoid origin are injected into and later harvested from the blood of the host animal. The xenograft cells are separated from the host blood cells and preserved. Preserved xenograft cell aggregates are embedded and processed using routine histological methods well known to those of ordinary skill in the art.

Referring again to FIG. 1, the xenograft tissue is grown to the desired extent and is removed surgically 13 from the host to be preserved in a fixative 14 and further processed. Fixation is performed in any appropriate manner consistent with routine histology procedures, which are well-known to those of ordinary skill in the art. After the xenograft tissue has been appropriately fixed, it is then embedded in an embedding medium such as paraffin wax 16. This may be accomplished using any routine histological practice including automated tissue processing equipment 15. The embedded xenograft control sample 16 is then apportioned for testing. Typically, apportioning is cutting thin-sections using a microtome or similar instrument and floating the sections onto a glass slide 17, onto which the sections are adhered by baking or other routine histology lab method. Another example of portioning is to make a mono-layer smear of xenograft tissue onto a glass slide, the details of which are well-known to one of ordinary skill.

At this point, the xenograft block could be used to cut tissue sections from that would be useful as controls for staining, but since the xenografts are heterogeneous, they must be screened first. The more optimal method is to create a xenograft “multiblock” to cut sections from. The receiving multiblock is simply a paraffin blocks with paraffin cores removed to make a set of empty receiving wells for embedded control cores to be placed in. Portions of the xenograft tissue are selected for their desirable characteristics, and then cores from the desirable areas of the xenograft are removed and placed into the multiblock wells.

The first step is to “map” the xenograft. This requires that the mounted thin-section of the xenograft tissue be analyzed and tested for ‘desirable characteristics’ to determine which areas or portions have acceptable test results. ‘Desirable characteristics’ are determined by but not limited to such testing as H&E, IHC, ISH and SS which elucidate which areas of the xenograft tissue are best for use in the xenograft control slide. ‘Desirable characteristics’ would include but not be limited to such qualities as a lack of necrosis, correct morphology, areas of xenograft tissue with minimal host tissue present, optimal fixation, and appropriate staining, whether with a chemical dye or by using an enzyme-based colorimetric assay such as IHC or ISH. Examples of appropriate staining are a positive ISH signal for HPV in HeLa xenograft tissue or 3+ positive IHC staining for c-kit in GIST 889 xenograft tissue or negative (0+) IHC staining for her-2 in MCF7 xenograft tissue.

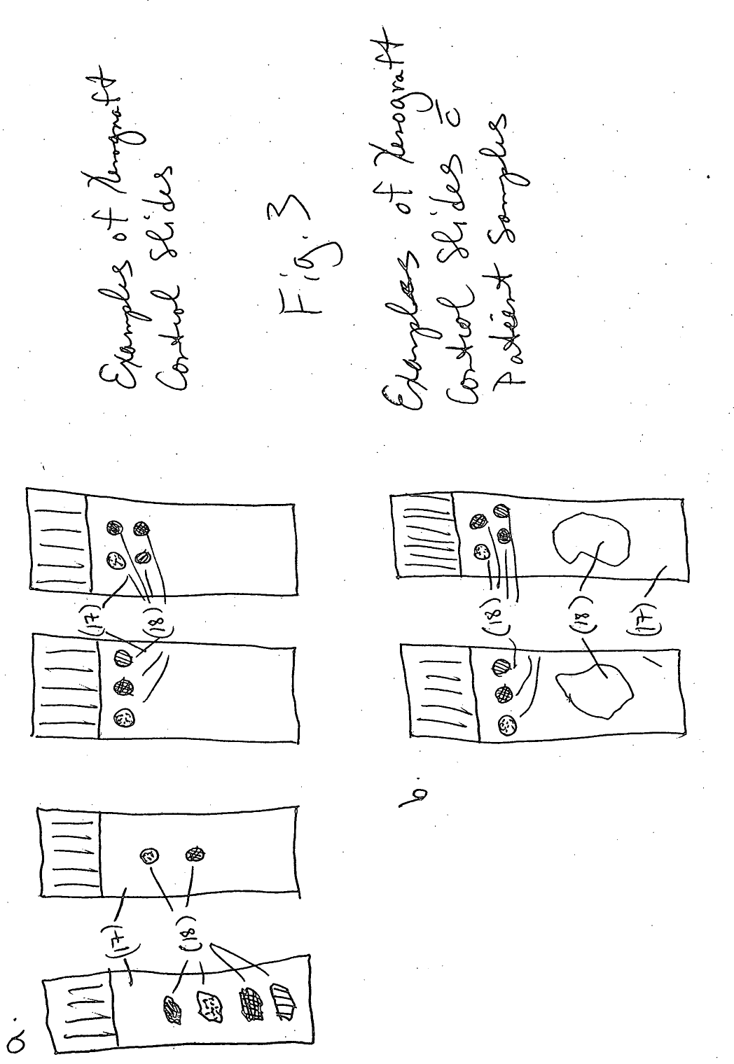

With respect to FIG. 2, once these desireable areas of the xenograft are identified, a cored xenograft sample 18 was taken to be placed into the pre-bored receiving paraffin block 20. Xenograft sample cores were created either manually or using instrumentation such as a microarray apparatus by using a bore 19 of defined size to cut into and through the embedded xenograft tissue sample. The bore may be variously shaped, of a range of diameters and made of metal, plastic or other hard material which would allow effective penetration of the mounting medium. The xenograft sample core was transferred into the pre-bored receiving block to a defined well. Multiple cores or small pieces may be placed into this prepared receiving block (multiblock) 17 so that the locations and identification of the xenograft sample cores is known. The final step usually involves heating the multiblock to soften the paraffin enough so that the cores are melted into place, thereby creating a uniform multiblock.

These cores provide a range of characteristics that will facilitate evaluation of the test results. For example, the multiblock can be composed of several corings which have a range of IHC staining intensities and which define the range of expected results for a given test.

As shown in FIG. 3, to create a xenograft control slide a section was cut from the xenograft multiblock and mounted on a slide 17. FIG. 3a shows four different microscope slides having xenograft controls 18 arranged upon them. The first slide shows four different controls arranged on the slide in a longitudinal array. The second slide is similar to the first but shows two longitudinal control sections. The third slide shows three controls arranged at the top of the slide to allow room on the slide for a patient sample, and the fourth is similar to the third but uses four controls. Use of the third and fourth slides in conjunction with a patient sample provides a good “on-board” control that is as close to the ideal as possible. The xenograft control slides also facilitate determination of the rate of incidence of false positive and false negative test results.

Actual testing conditions include deparaffinization, antigen retrieval or cell conditioning in order to prepare the sample for testing. Diagnostic testing such as IHC and/or ISH is performed on the test sample and the xenograft control samples. The testing can be performed manually or on an automated system. The xenograft control slide can be run separately but identically to the patient sample as shown in FIG. 3a, or the patient sample can be mounted onto the same slide as the xenograft control as shown in FIG. 3b and tested together under the same conditions. The results of each sample are then compared using microscopic examination by a person trained in the art of pathology or histology.

The xenograft control samples are pre-defined and characterized so that any test results deviating from the expected would indicate a failure with the diagnostic test. For example, a lack of staining or reaction of the xenograft control, when it is known to be positive, would indicate a false negative result. Conversely a staining event or reaction in a known negative sample (0+) would indicate a false positive result. The results of the xenograft control slide allow one of ordinary skill in the art to evaluate the performance and validity of the diagnostic test and to determine whether the results are acceptable or the test needs to be repeated.

Another use of the xenograft control result would be to compare the intensity or type of result present in the control with that of the test sample. An example of this would be where the control samples have a range of IHC staining intensities present in each of four samples (e.g. 0, 1+, 2+, 3+). The patient sample staining intensity is compared to this range and rate of response is determined. The level of intensity is used to diagnose and/or determine prognosis and treatment of the pathology presented. For example, it is well known that in a sub-population of breast cancer patients, if there is found overexpression of the Her-2/neu protein in the cellular membrane, that is an indication that HERCEPTIN® therapy may be indicated. In an IHC test using the anti-Her-2 CB 11 antibody from Ventana Medical Systems (Tucson, Ariz.) if a patient sample shows a response of 3+ in a Her-2/neu assay, the patient will likely be a candidate for HERCEPTIN therapy. However, if the patient sample shows only a 0 or 1+ reading when compared to the control, then the threshold may not be met.

The xenograft control sample has great advantages over conventional control samples by providing a control sample in a form more native or natural than other manufactured control samples and thus more representative of patient material. The xenograft control also has the advantage of being highly characterized, homogeneous in composition, reproducible and can be manufactured in a routine fashion over time. While patient samples have been used as control samples they are limited in quantity and/or occurrence, can lack homogeneity and are less well characterized. There are also restrictions arising from patient consent and confidentiality requirements.

### EXAMPLES

The following examples are meant to illustrate the embodiments of the invention but are not intended to limit it in any way.

### Example 1

### - Growing and Characterization of HPV Xenograft Tissue Controls

An example of a xenograft control slide for an in situ HPV assay is a slide containing four cores of xenograft tissue which contain four different amounts of integrated HPV DNA: CaSki (250-500 copies of HPV Type 16), HeLa (25-50 copies, HPV Type 18), SiHa (1-2 copies, HPV Type 16) and T-24 (0 copies). The xenograft control slides are made by growing transformed cervical (CaSki, HeLa and SiHa) and bladder carcinoma (T24) cell lines using standard laboratory practices (as described in R. I. Freshney, Culture of Animal Cells, 4^{th} ed., Wiley-Liss, New York, 2000). The CaSki cells were grown in RPMI-1640 medium plus L-glutamine (MediaTech, Cat.# 10-040-CV) supplemented with 10% Fetal Bovine Serum (FBS) (Gibco, Cat.# 16000-044), 10 mM HEPES (Hyclone, Cat.# SH30237.01), 1 mM sodium pyruvate (MediaTech, Cat.#25-000-CI), 1.5 g/L sodium bicarbonate (Gibco, Cat.#25-035-CI) and 1% penicillin streptomycin (Gibco, Cat.# 15140-122). The HeLa cells were grown in Eagle's Minimal Essential medium plus L-glutamine (MediaTech, Cat.# 10-010-CV) supplemented with 10% FBS, 1 mM sodium pyruvate, 0.1 mM non-essential amino acids (Hyclone, Cat.# SH30238.01), 1.5 g/L sodium bicarbonate and 1% penicillin streptomycin. The SiHa cells were grown in Eagle's Minimal Essential medium plus L-glutamine supplemented with 10% FBS, 1 mM sodium pyruvate, 0.1 mM non-essential amino acids, 1.5 g/L sodium bicarbonate and 1% penicillin streptomycin. The T-24 cells were grown in McCoy's 5a medium plus L-glutamine (MediaTech, Cat.# 10-050-CV) supplemented with 10% FBS and 1% penicillin streptomycin. Cell lines were maintained under humidified conditions containing 5% CO₂ at 37° C. Cells are grown to 90-100% confluence. Trypsin (0.25%) with EDTA (Gibco, Cat.# 25200-072) was added to detach adherent cells. Cells (90-100% viability) are counted and resuspended at a concentration of 10×10⁶ cells/200 μl sterile 1×Conc. Dulbecco's PBS (Hyclone, Cat.# SH30028).

A SCID mouse, developed and maintained as described in Paine-Murrieta, et al Cancer Chemother. Pharmacol. 40: 209-214 (1997), is injected subcutaneously (s.c.) with tumor cells (200 μl) in the flank. Following tumor development, the volume (cm²) of the tumors are estimated in accordance with the formula (tumor width²×tumor length/2). When the tumors reach an approximate size of 500 mm², the mice are sacrificed and the tumors removed surgically. The tumors are placed in 10% neutral buffered formalin (VWR, Cat. # VW3239) for 24 hours at room temperature (RT). The fixed tumors are placed into a tissue cassette and processed into paraffin using standard histological methods as described in Carson, F., Histotechnology A Self-instructional Text, ASCP Press, Chicago, Ill. (1997).

The paraffin-embedded xenograft tissue was thin-sectioned onto a glass slide (VWR, Cat. #48311-703) and characterized for use. A slide was stained using H&E to determine morphology type and location of any necrotic areas. In addition, slides were stained using an automated in situ HPV assay, two each with an HPV positive and negative probe, to determine how the xenograft tissue stains. In addition, the xenograft tissues were stained with a probe to alu, a highly repeated DNA sequence found in humans to confirm which areas of the tumor are human in origin and which are mouse. Using the variously stained slides, areas are identified for coring on the H&E slide.

### Example 2

### Multiblock Construction

To create a xenograft control multiblock, a recipient block is first made using a microarrayer instrument. The recipient block was placed face up into the recipient block holder. Using the tissue microarrayor and a 2 mm core, the punch was positioned in the precise area where the first piece of tissue is to be embedded. A core was removed from the receiving block. The coring process was repeated until four paraffin cores were removed from the recipient bock. The recipient block was removed from the microarrayor and replaced with the xenograft tissue block to be cored. Using the H&E slide, the xenograft tissue was lined up with the punch to the precise spot on the xenograft tissue block to be cored. The xenograft tissue block was cored and released from the punch. The xenograft tissue block was replaced with the recipient block, and the recipient block hole lined up to the punch with the xenograft tissue core. The xenograft tissue core was ejected into the recipient block hole. These steps are repeated until all four tissue samples have been placed into their designated areas (per the block map) in the recipient block to form the multiblock. The completed multiblock was partially melted to allow the block and cores to fuse into a single entity. The fused multiblock was allowed to harden at room temperature (RT) and was ready for thin-sectioning. Thin-sections are prepared on glass slides using standard methods as described in Carson, F. (1997).

### Example 3

### Xenograft Controls Run with Ventana's HPV Assay

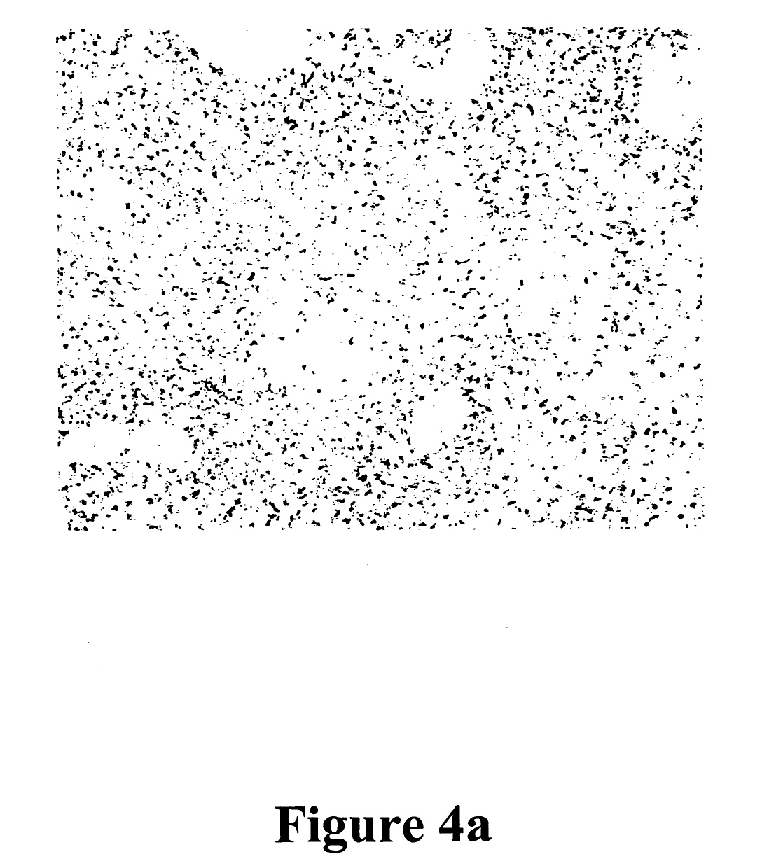

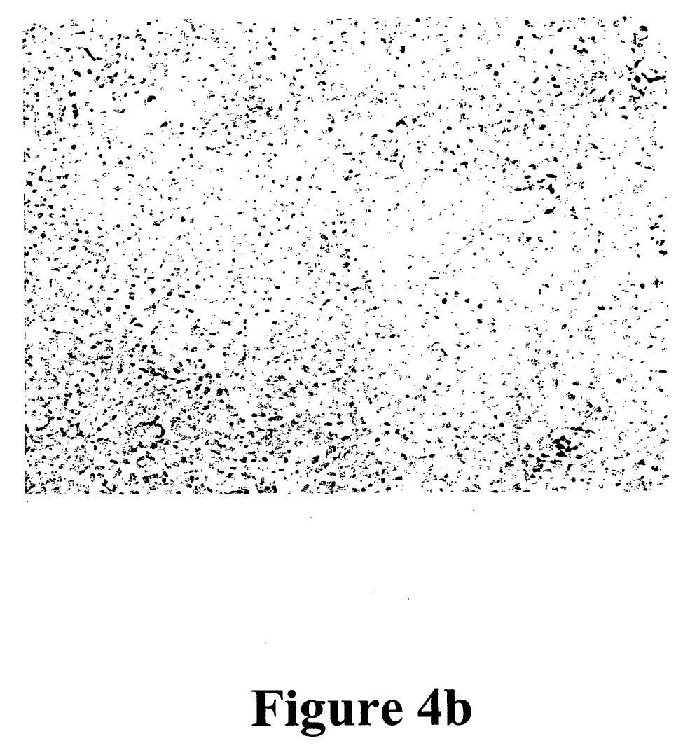

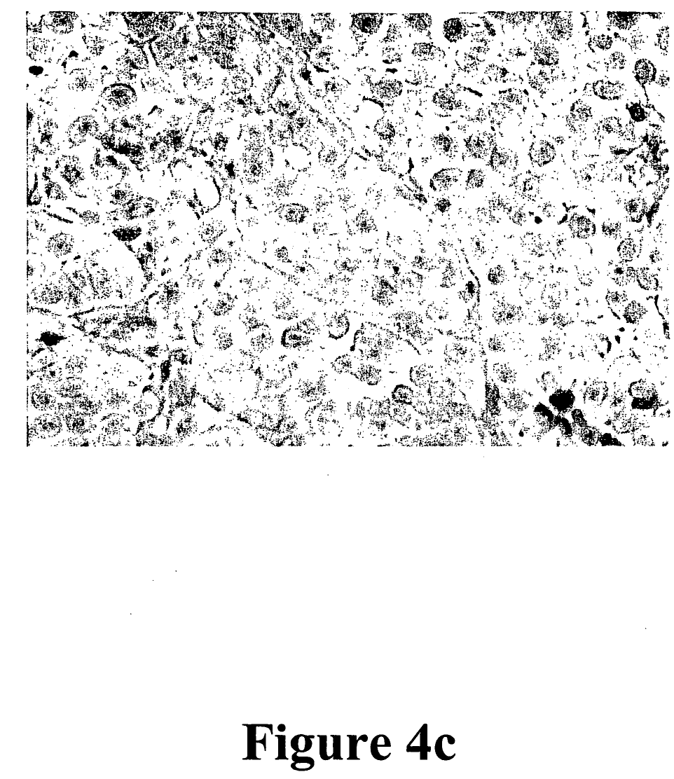

FIGS. 4A-C are microphotographs of three HPV xenograft control slides tested with an ISH assay using an HPV DNA probe. Using a BENCHMARK® series autostainer, (Ventana Medical Systems, Tucson, Ariz.) slides containing HPV xenograft controls were stained using INFORM® HPV Family 16 DNA Probe (PN 780-2838), detected with ISH iView Blue Detection Kit (PN 760-092) and counterstained with ISH Red Counterstain. The HPV Family 16 probe is a cocktail of DNA probes with specificity for high-risk HPV types 16, 18, 31, 33, 35, 39, 45, 51, 52, 56, 58 and 66. CaSki (FIG. 4a) and HeLa (FIG. 4b) xenograft tissues exhibit positive staining (blue punctuate pattern) due to nucleus-integrated HPV DNA. Negative staining (no blue punctuate pattern) is seen in the T24 (FIG. 4c) xenograft

### Example 4

### Xenograft Controls Run with Ventana's Her-2/Neu Assay

Another example of a xenograft control slide for an immunohistochemistry Her-2 assay is a slide containing four cores of xenograft tissue which contain four different levels of Her-2 protein in the following transformed human breast carcinoma cell lines: BT474 (3+ staining intensity), transformed carcinoma (2+ staining intensity), ZR-75-1 (1+ staining intensity) and MCF7 (0+ staining intensity), all available from ATCC, Manassas, Va. The xenograft control slides were made by growing the transformed breast carcinoma cell lines using standard laboratory practices (as described in R. I. Freshney, 2000). The BT-474 cells were grown in Hybri-Care medium plus L-glutamine (ATCC, Cat.# 46X) supplemented with 10% FBS and 1% penicillin streptomycin. The ZR-75-1 cells were grown in RPMI-1640 medium plus L-glutamine supplemented with 10% FBS, 10 mM HEPES, 1 mM sodium pyruvate, 1.5 g/L sodium bicarbonate and 1% penicillin streptomycin. The MCF7 cells were grown in Eagle's Minimal Essential medium plus L-glutamine supplemented with 10% FBS, 0.1 mM non-essential amino acids, 20 ul/ml insulin and 1% penicillin streptomycin. Cell lines were maintained under humidified conditions containing 5% CO₂ at 37° C. Cells are grown to 90-100% confluence. Trypsin (0.25%) with EDTA was added to detach adherent cells. Cells (90-100% viability) are counted and resuspended at a concentration of 10×10⁶ cells/200 μl sterile 1× Conc. Dulbecco's PBS. The tumors were grown and processed the same as above and the xenograft multiblock was assembled in the same manner as described above.

The Her-2 xenograft control slide allows the user to control for sensitivity and expression level (range of staining intensities), determine treatment (treatment with HERCEPTIN is indicated in cases with a staining intensity greater than 2+), disease prognosis (amplified Her-2 indicates an aggressive carcinoma that is resistant to many treatments) and over-staining (negative sample). If any of the xenograft control samples do not stain at the prescribed intensity, those of ordinary skill in the art are alerted to potential misdiagnosis of the patient sample. The type of failure can also be an indication of the source of malfunction in the diagnostic test which facilitates troubleshooting and correction of the malfunction. For example, if the negative sample were to appear positive, this might indicate that there was a rinsing problem with the test or that the incorrect type of test had been used.

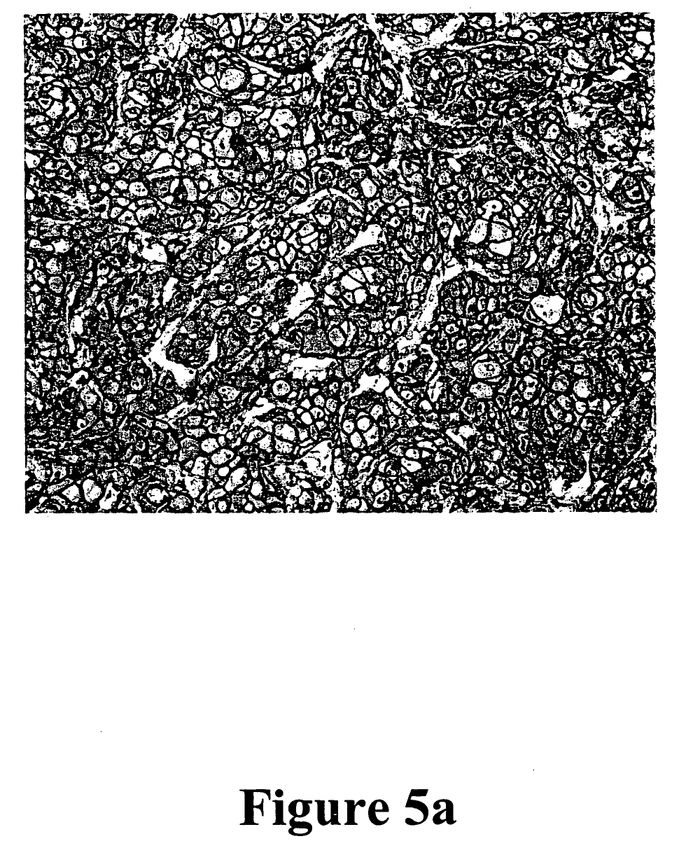

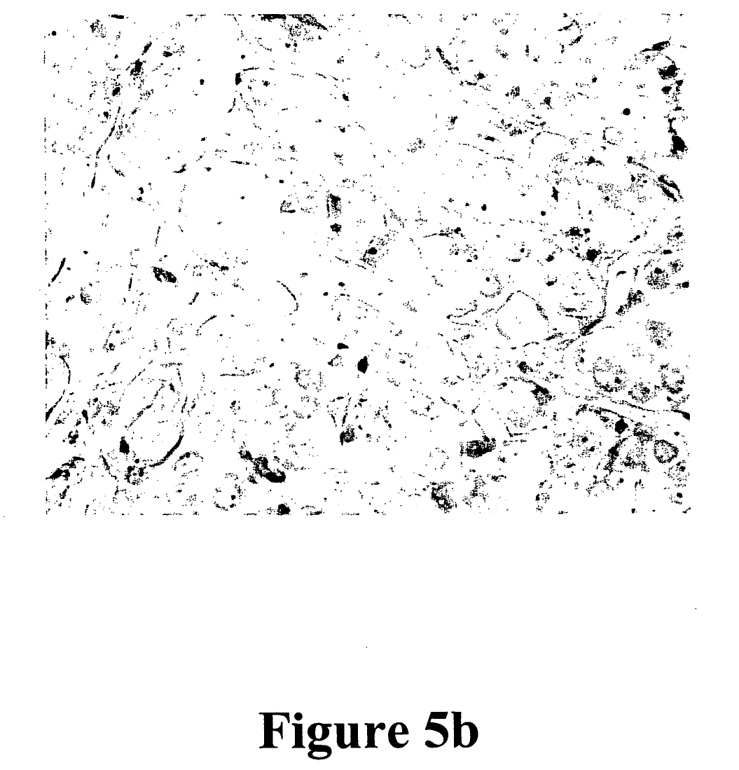

FIG. 5 is a photomicrograph of a BT474 xenograft control slide tested alongside an IHC assay using anti-Her-2 antibody 4B5 (Ventana). Using a BENCHMARK® series autostainer (Ventana), slides containing Her-2 xenograft controls were stained using PATHWAY® anti-Her-2 antibody 4B5 (Ventana), detected with iView DAB Detection Kit (Ventana PN 760-091) and couterstained with Hematoxylin. FIG. 5a shows that BT-474 xenograft tissue exhibits a positive stain (brown/DAB cytoplasmic membrane). FIG. 5b shows negative staining (i.e. no brown pattern) is seen in the MCF7 (b) xenograft tissue.

### Example 5

### Three-in-One HPV Control Slides

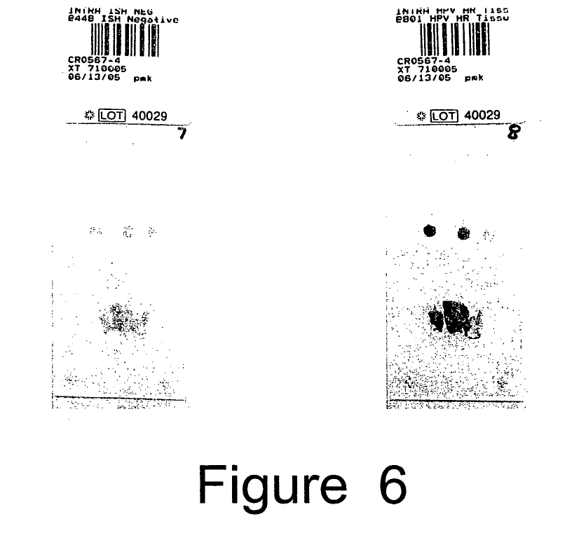

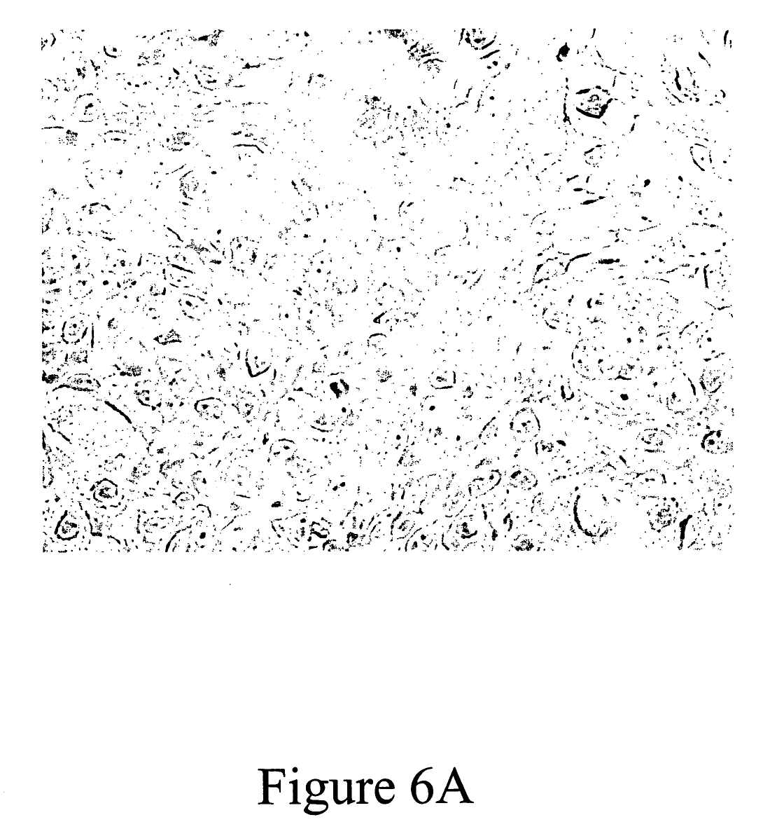

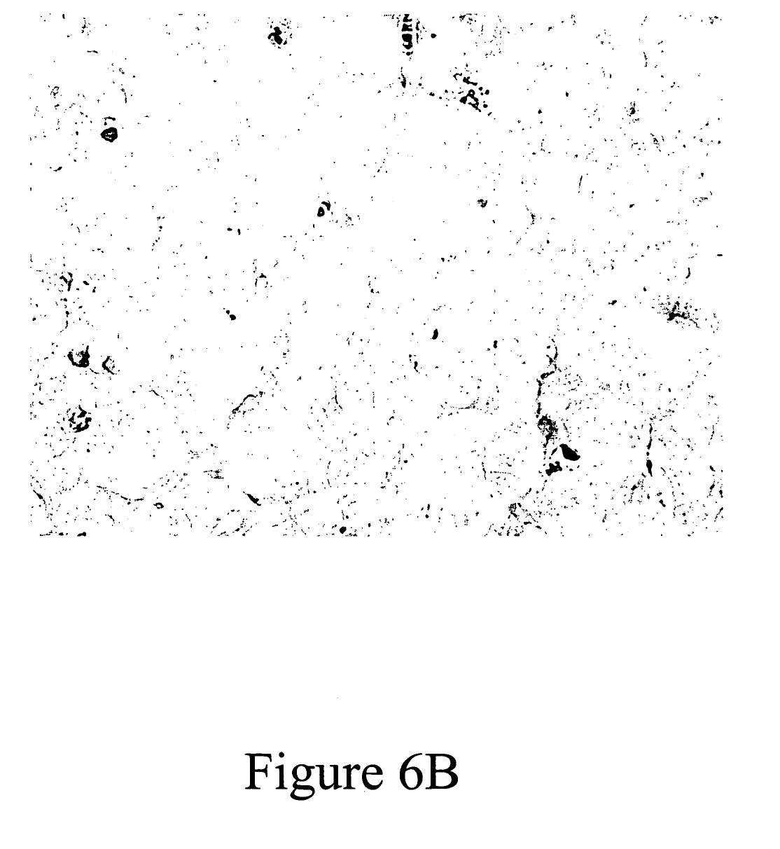

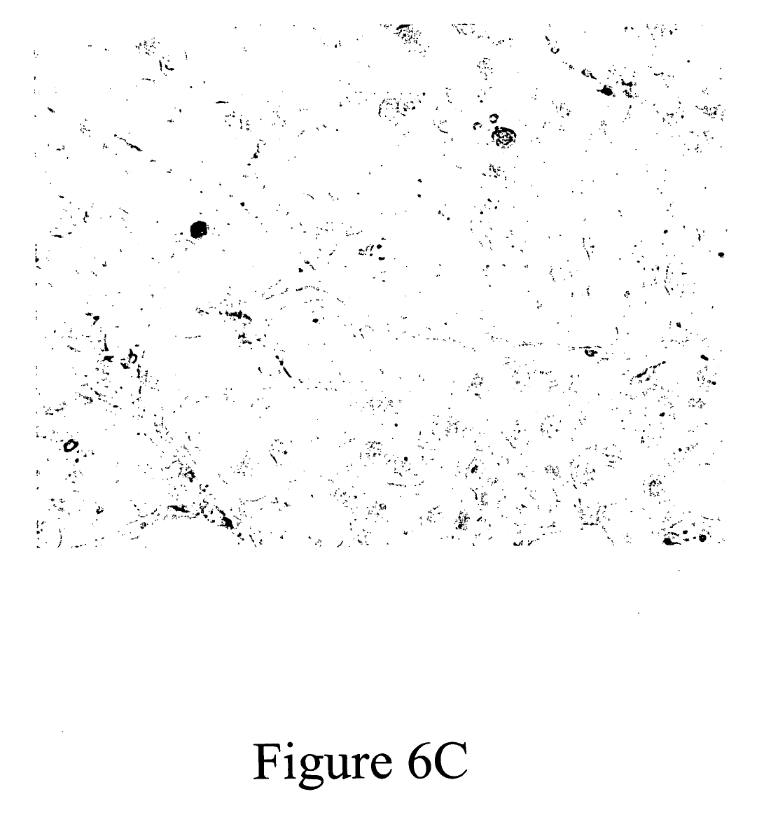

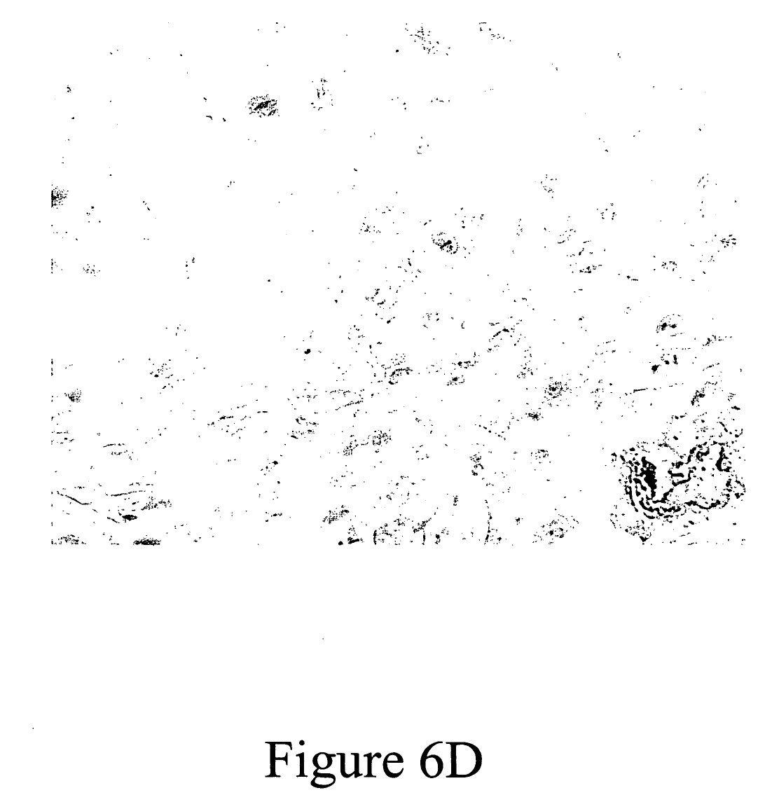

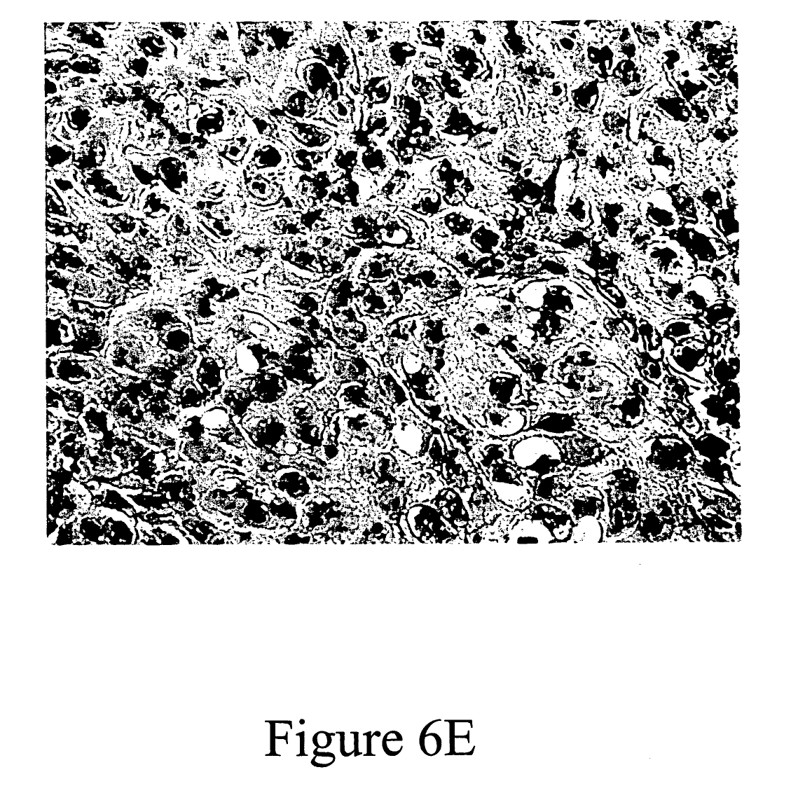

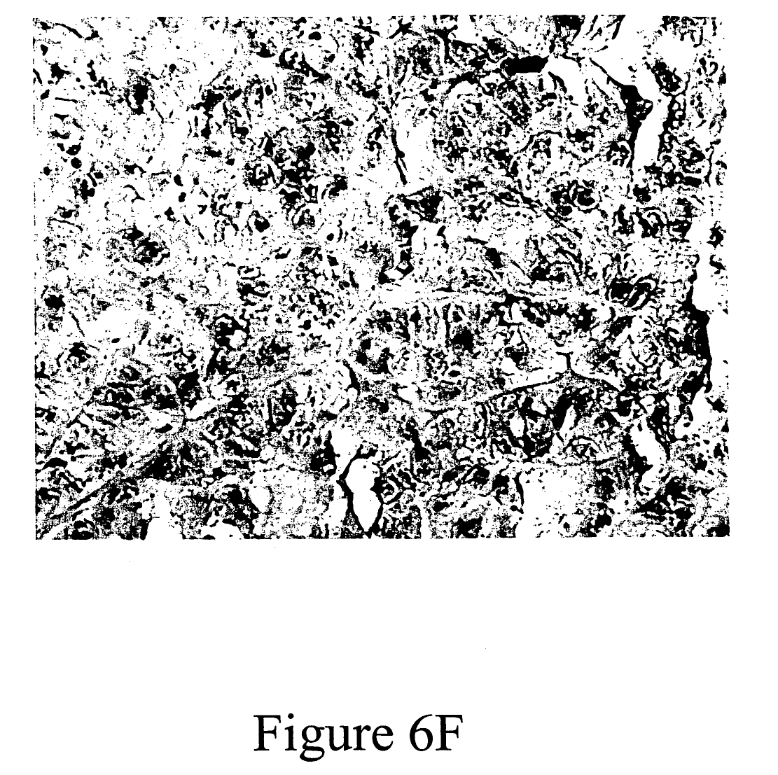

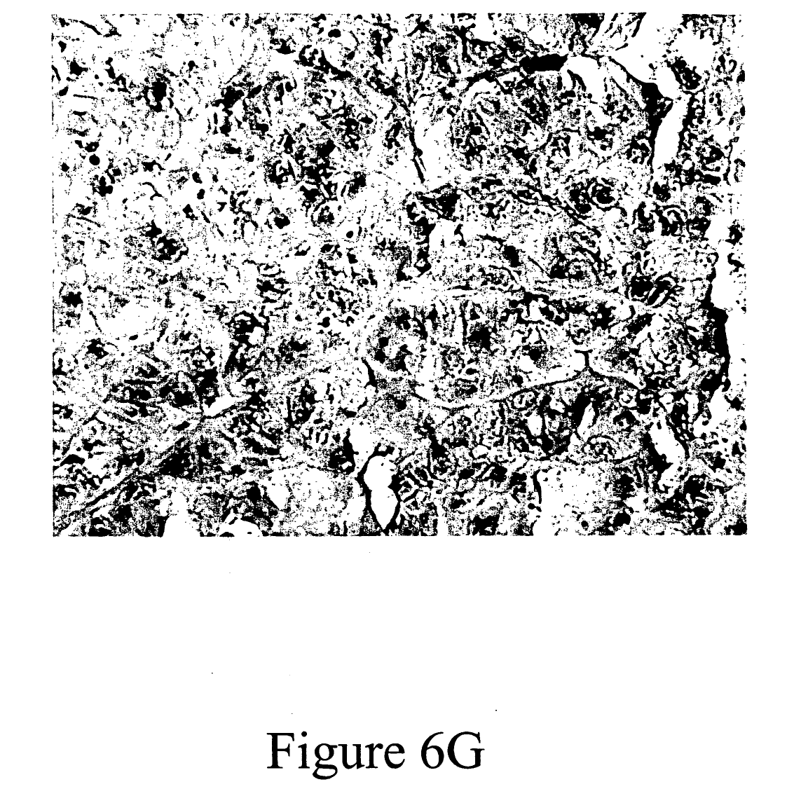

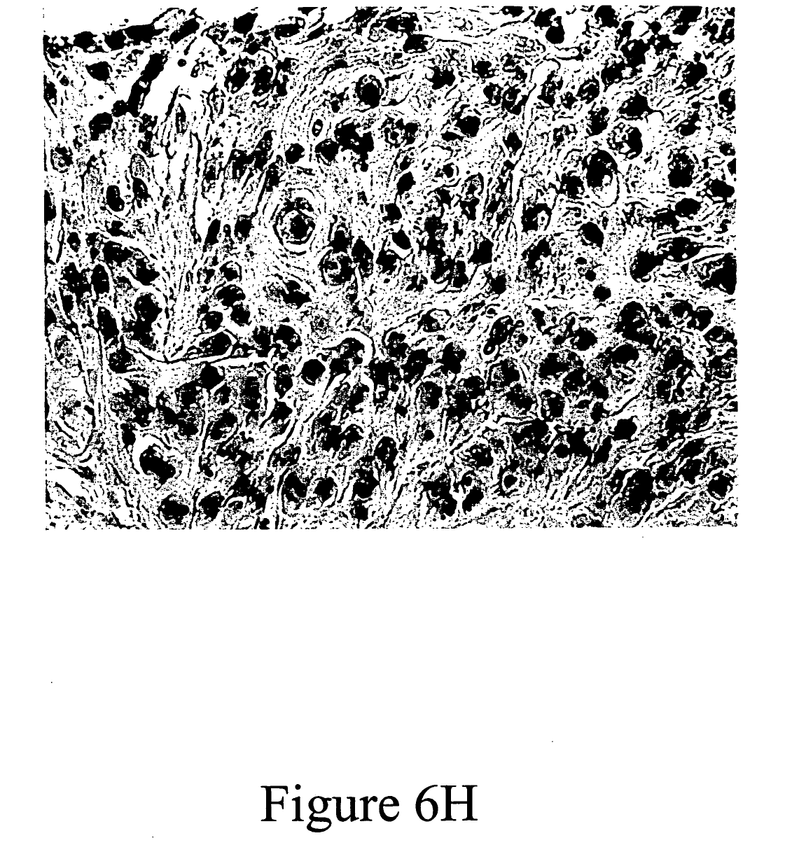

FIG. 6 is a photograph of two stained three-in-one HPV control slides including actual patient samples. The xenograft control cores, located at the top of the slide, are comprised of CaSki (far left, 250-500 copies), HeLa (middle, 25-50 copies) and C33a (far right, 0 copies) xenografts. The patient test sample (surgically removed cervical tissue) is located below the cores in the center of the slide. The left slide is stained with negative HPV ISH probe (buffer only) and counterstained with Nuclear Fast Red. The C33a cell line is a human cervical cancer line (ATCC# HTB-31) that is know to be HPV negative by PCR.

FIGS. 6A-D show close-ups at 40× magnification of the top three control cell lines and the sample, in consecutive order, stained with the ISH Negative Control probe. All samples, including the patient's, have no blue punctuate staining indicating that the test performed accurately and the system is functioning as expected.

FIGS. 6E-H show close-ups at 40× magnification of the top three control cell lines and the sample, in consecutive order, stained with the HPV Family 16 DNA Probe. FIG. 6E (Caski) demonstrates a high amount of blue punctuate staining indicating positive HPV DNA, as expected. FIG. 6F (HeLa) demonstrates a medium amount of blue punctuate staining indicating HPV DNA present from 25-50 copies per cell. FIG. 6G (negative control, C33A) has no blue punctuate staining as is expected in the negative cell line. These results indicate that the HPV Family 16 test and BenchMark® series autostainer have performed as expected. The patient test sample also contains blue punctuate staining in the epithelial cells of the cervix indicating infection with HPV.

These slides were created using a BENCHMARK® series autostainer, (Ventana Medical Systems, Tucson, Ariz.) The 3 in 1 HPV xenograft control slides were stained using either INFORM® HPV Family 16 DNA Probe (PN 780-2838) or ISH Negative Control Probe, detected with ISH iView Blue Detection Kit (PN 760-092) and counterstained with ISH Red Counterstain. The HPV Family 16 probe is a cocktail of DNA probes with specificity for high-risk HPV types 16, 18, 31, 33, 35, 39, 45, 51, 52, 56, 58 and 66. The ISH Negative Control Probe is composed of probe buffer only, no DNA.

### Example 6

### Three-in-One Her2 Control Slides

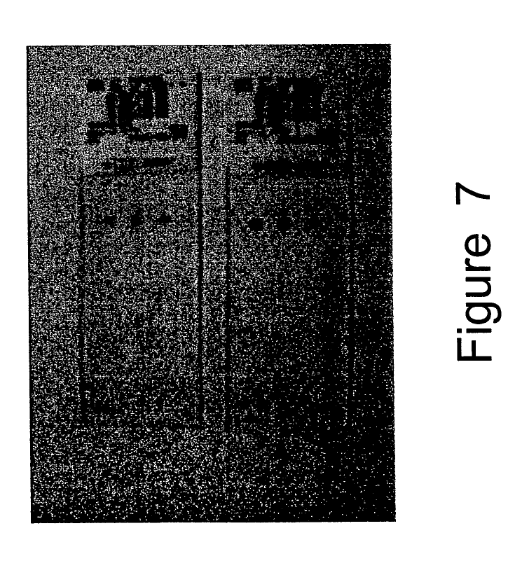

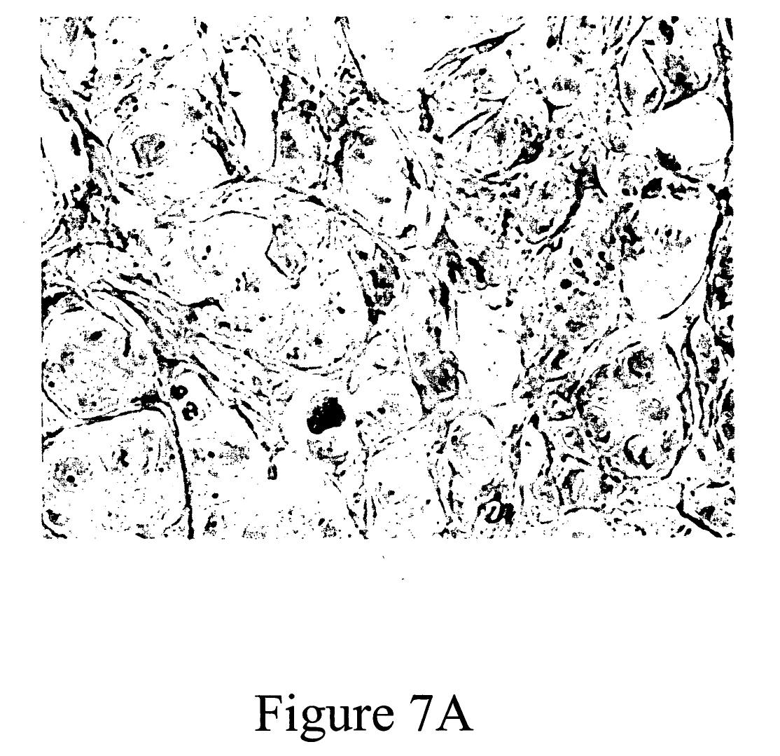

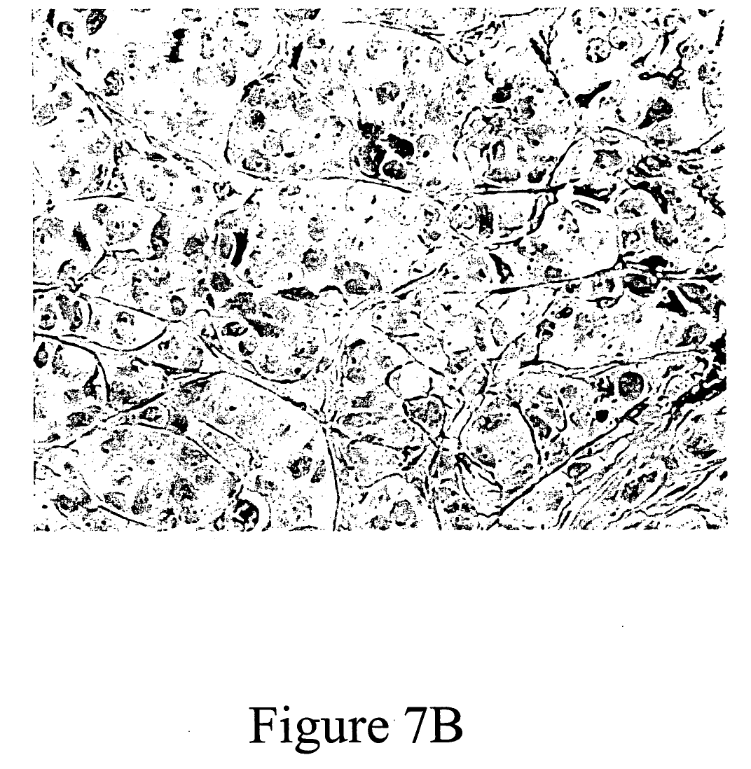

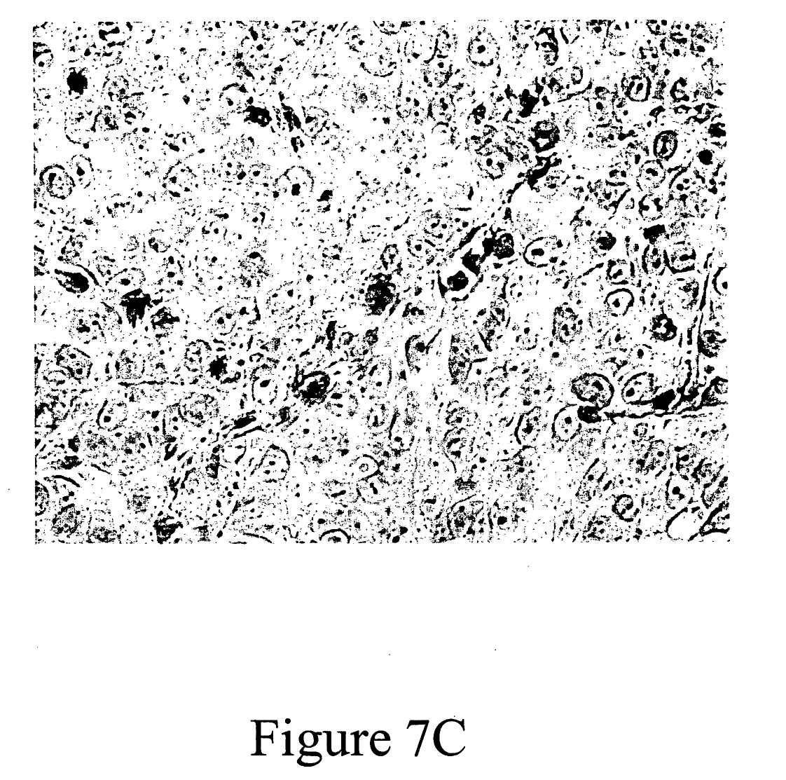

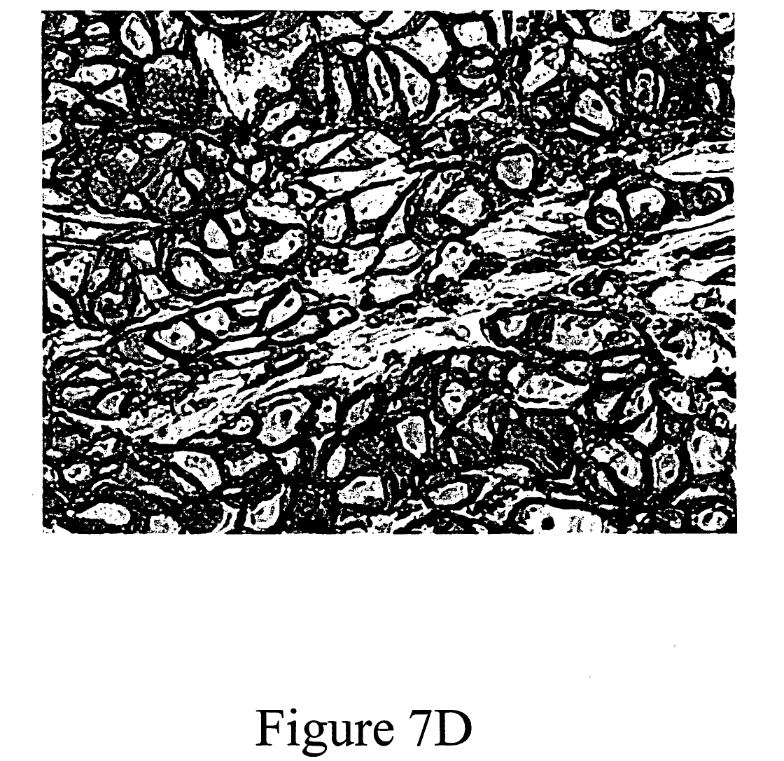

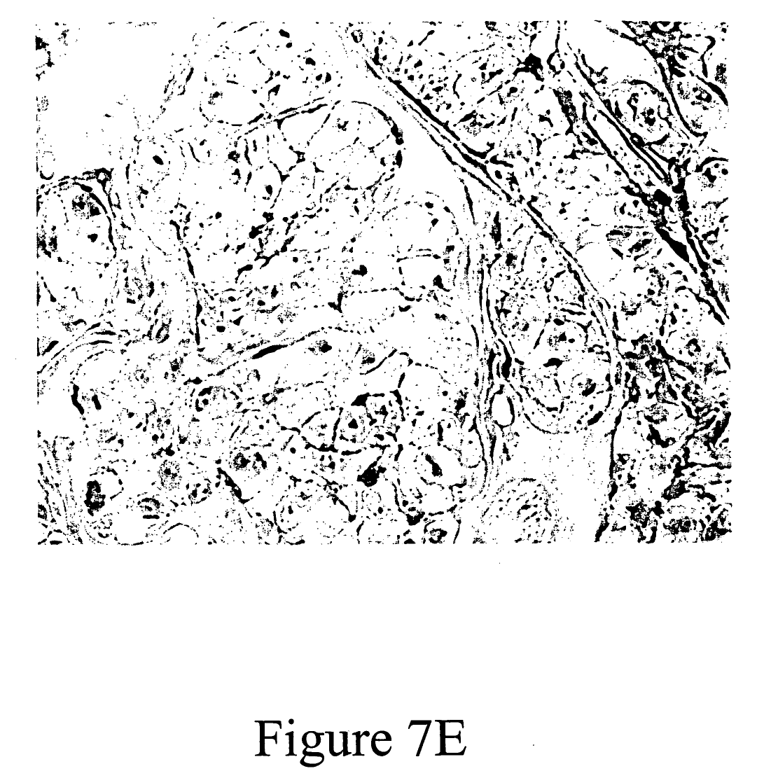

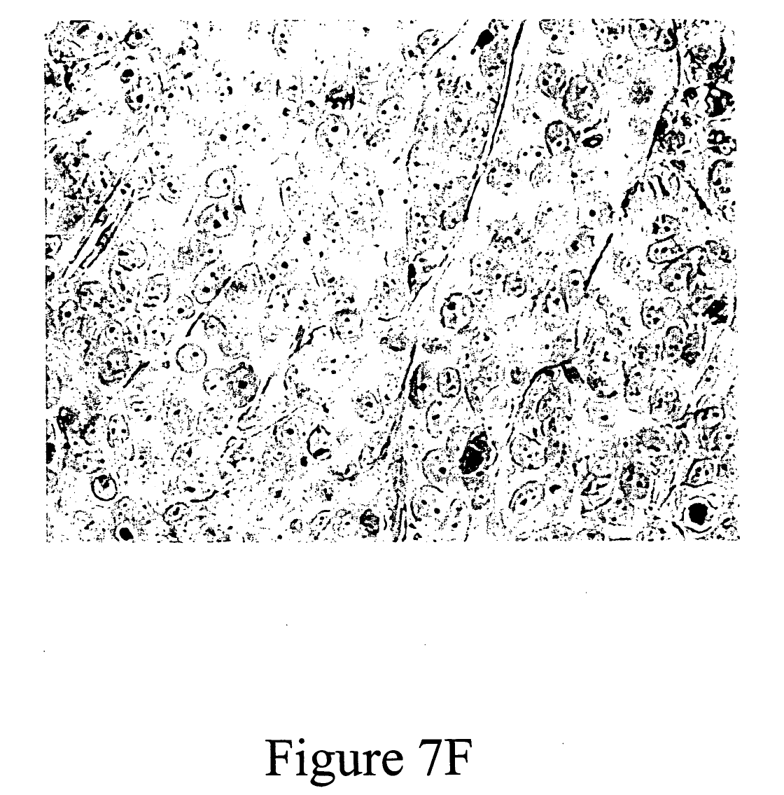

FIG. 7 is a photograph of two stained three-in-one HER2 control slides. The top slide is stained with rabbit negative control antibody (rabbit IgG) (Ventana P/N 760-1029) and counterstained with (Hematoxylin and Bluing reagent). The bottom slide is stained with anti-Her2 rabbit monoclonal antibody 4B5 (Ventana PN 790-2996). FIGS. 7A-C are 40× magnification photomicrographs of the three xenograft control cell lines from the top slide. FIGS. 7D-F are 40× magnification photomicrographs of the same three cell line controls, this time exhibiting positive staining.

The xenograft control cores, located at the top of the slide, are comprised of BT-474 (far left), ZR75-1 (middle) and MCF7 (far right) xenografts. The top slide is stained with Rabbit Negative Control antibody (rabbit IgG) and counterstained with Hematoxylin and Bluing reagent. The bottom slide is stained with rabbit anti-HER2 (4B5) and counterstained with Hematoxylin and Bluing reagent.

FIGS. 7A-C show close-ups at 40× magnification of the three control cell lines in consecutive order, stained with the Rabbit Negative Control antibody. All samples, have no brown staining indicating that the test performed accurately and the system is functioning as expected.

FIGS. 7D-F show close-ups at 40× magnification of the three control cell lines, in consecutive order, stained with Rabbit anti-Her2 monoclonal antibody (4B5). FIG. 7D demonstrates a high amount of brown nuclear membrane staining indicating positive HER2 protein presence. FIG. 7E demonstrates a low amount of brown nuclear membrane staining indicating Her2 protein at a lesser level. FIG. 7F has no brown staining as is expected in the negative cell line. These results indicate that the HER2 test and BenchMark® series autostainer have performed as expected.

These slides were created using a BENCHMARK® series autostainer, (Ventana Medical Systems, Tucson, Ariz.) The 3 in 1 HER2 xenograft control slides were stained using either Rabbit anti-HER2 (4B5) (PN 780-2838) or Rabbit Negative Control antibody (P/N 760-1029), detected with iView DAB Detection Kit (PN 760-091) and counterstained with Hematoxylin and Bluing Reagent. The anti-HER2 (4B5) antibody is a rabbit monoclonal antibody, specific for the HER2 protein that is diluted to appropriate concentration in a proteaneceous Tris-based buffer. The Rabbit Negative Control antibody is a polyclonal antibody from non-immunuzed rabbits that is diluted to appropriate concentration in a proteaneceous tris-based buffer.

While the invention has been described with reference to an illustrative embodiment, it will be understood by those skilled in the art that various changes, omissions and/or additions may be made and equivalents may be substituted for elements thereof without departing from the spirit and scope of the invention. In addition, many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims. Any publications or patents referenced herein are incorporated by reference in their entirety.

We claim:

1. A method of making a tissue control substrate, comprising:
growing a xenograft from a mammalian transformed cell line in a host animal;
removing the xenograft from the host animal; and
processing the xenograft thereby embedding the xenograft tissue in an embedding medium.

2. The method of claim 1 wherein the tissue control substrate comprises a microscope slide.

3. The method of claim 3 wherein the mammalian transformed cell line is selected from the group consisting of SiHa, Caski, HeLa, T24, BT474 and MCF7.

4. The method of claim 1 wherein the host animal is an immunodeficient mouse.

5. The method of claim 4 wherein the immunodeficient mouse is a SCID mouse.

6. The method of claim 1 additionally comprising the step of identifying and removing any host tissue from the xenograft.

7. The method of claim 1 wherein processing the xenograft comprises embedding the xenograft in a paraffin-based embedding medium.

8. The method of claim 1 wherein said method additionally comprises the step of affixing the embedded xenograft sample onto a substrate.

9. A method of using a xenograft as a control tissue for histology, comprising:
staining both a patient specimen and a xenograft-derived control sample under substantially similar staining conditions; and
assessing the staining outcomes of the two to determine whether the stain was effective for the patient specimen.

10. The method of claim 9 wherein the step of staining comprises contacting both the patient specimen and the xenograft control samples with a stain selected from the group consisting of in situ hybridization probes, immunohistochemical stains and special stains.

11. The method of claim 9 wherein substantially similar staining conditions comprises staining both the patient specimen and the xenograft control on the same slide.

12. The method of claim 9 wherein substantially similar staining conditions comprises staining both the patient specimen and the xenograft control during the same run or batch.

13. The method of claim 9 wherein determining whether the staining was effective for the patient specimen comprises comparing the actual staining of the xenograft control with the expected staining of the xenograft control.

14. The method of claim 13 wherein the degree of staining is assessed on a 0 to 3+ scale.

15. A xenograft-derived control slide for histochemical use, comprising:
at least one xenograft control sample prepared for histological use; and
a sample slide upon which the at least one xenograft control sample is affixed.

16. The xenograft-derived control slide of claim 15 wherein the at least one xenograft control sample comprises both at least one negative xenograft control sample and at least one positive xenograft control sample.

17. The xenograft-derived control slide of claim 15 wherein the at least one xenograft control sample comprises at least one negative xenograft control sample, and a plurality of positive xenograft control samples.

18. The control slide of claim 17 wherein the positive xenograft control samples comprise a Caski xenograft.

19. The control slide of claim 17 wherein the positive xenograft control samples comprise a HeLa xenograft.

20. The control slide of claim 17 wherein the negative xenograft control samples are selected from the group consisting of C33A and T24 cell lines.

21. The control slide of claim 17 wherein the positive xenograft control samples comprise a BT-474 xenograft.

22. The control slide of claim 17 wherein the positive xenograft control samples comprise a ZR75-1 xenograft.

23. The control slide of claim 17 wherein the negative xenograft control samples comprise a MCF7 xenograft.
